(19) United States
(12) Patent Application Publication
(10) Pub. No.: US 2021/0358618 A1
(43) Pub. Date: Nov. 18, 2021

(54) SYSTEM, APPARATUS AND METHOD FOR FACILITATING PERSONAL ENGAGEMENT AND ROAMING ACCESS TO CARE

(71) Applicant: AllyAlign Health, Inc., Glen Allen, VA (US)

(72) Inventor: David Michael Crocker, Nashville, TN (US)

(21) Appl. No.: 17/319,354

(22) Filed: May 13, 2021

Related U.S. Application Data
(60) Provisional application No. 63/024,148, filed on May 13, 2020.

(51) Int. Cl. G16H 40/67; G16H 80/00

(52) U.S. Cl. CPC G16H 40/67; G16H 80/00

(57) ABSTRACT

A device, system and method facilitate personal engagement and roaming access to care for individuals in need. In various embodiments, the device includes a movable stand, a support pole mounted to the stand and interactive care and personal engagement facilitation devices secured to the support pole. In various embodiments, the system comprises one or more interactive care and personal engagement facilitation devices comprising a video camera, a video display and/or an interactive touch screen.

### CROSS-REFERENCE TO RELATED APPLICATIONS

The present application claims the benefit of and priority to U.S. Provisional Application No. 63/024,148, filed on May 13, 2020. The content of the foregoing application is hereby incorporated by reference herein in its entirety.

### TECHNICAL FIELD

The present disclosure relates generally to portable communications equipment and systems, and more particularly to a system, apparatus and method for facilitating roaming access to care and interactive engagement for individuals.

### BACKGROUND AND SUMMARY

Many types of remote communications devices and technologies exist. For example, video meeting services such as FaceTime™, Zoom™ and others permit face-to-face real-time video capabilities. Further, e-mail and telecommunications services permit people in different locations to communicate in real-time via written or spoken word. However, while mobile communications devices, telehealth services and video-conferencing software permit a certain level of interactive engagement for otherwise isolated individuals such as residents of senior living and other caregiving facilities, such users can find it challenging to interact with individuals and service providers with whom they need or desire to interact with for personal, professional, caregiving and other purposes. For example, otherwise isolated individuals may need to interact with family members when making decisions about their health, finances, care plan coverage and other matters. At the same time, such individuals may need to interact with professionals such as healthcare practitioners, attorneys, accountants and other caregivers and/or service providers. Further, such outside parties may not always be physically present during times of need for a variety of reasons.

There is currently no capability or system which can permit multiple users to interact with an otherwise isolated individual such as a resident or member of a senior living facility so as to provide the ability for the individual to simultaneously communicate with a health care provider, one or more family members and other individuals and systems as described herein for providing care and interactive engagement.

The present disclosure relates to a device, system and method that facilitates personal engagement and roaming access to care for individuals in need. In various embodiments, the device includes a stand mounted on lockable casters or other rolling devices to permit the stand to be moved along the ground or floor surface and locked in place upon arrival at a desired destination. A support pole can be mounted to the stand and provided with mounting hardware to support desired equipment at multiple heights along the pole. For instance, the mounting hardware can include one or more clamps securable to the pole, wherein various types of equipment can be secured to the clamps. Such equipment can include, for example, a camera mount for receiving a camera to capture images of a desired person or subject, one or more monitor shrouds or brackets to support a video monitor, tablet or similar devices, a power cord holder, one or more baskets or other retainers for personal items, a handle for moving the stand and other devices. In various embodiments, the pole is provided with openings in its outer wall for managing electrical and communications wiring, for example. A power supply may be provided and maintained on the stand or pole. Alternatively, wiring can be secured and connected to a suitable electrical power source.

One or more computing devices can be secured to the pole to provide a way for users to communicate with a wide variety of people and computing systems, either at the same time or at different times. The computing devices can be considered as interactive care and personal engagement facilitation devices as described herein. For instance, one monitor may connect the user virtually to a nurse practitioner whereas another monitor may show diagnostics or may connect the user virtually to one or more family members. Each monitor may be operated by one or more computing devices, wherein the computing devices contain necessary processing and memory capabilities and operate necessary software to implement desired functionalities such as described herein. In at least one embodiment, a single computing device operates multiple monitors. The system can be operated entirely remotely in various embodiments, such that no personnel must be present to control the use. Depending upon the implementation and the user's desires or needs, the user may employ a monitor to connect visually with a health plan coordinator, enrollment specialist or other professional service provider. The health plan coordinator, specialist or other service provider can remotely control the device(s) at the user's location. The user may also use one or more screens to view documentation, videos or other visual subject matter. The monitor(s) can be touch-sensitive so as to allow interaction with forms or other material through the touch of the screen. In various embodiments, the device can be employed and accessible by bed-bound residents of a senior living or other caregiving facility. The system and device may be used for telehealth, as a standalone health plan sales kiosk, as a computing infrastructure for completing health risk assessments or various other duties.

### BRIEF DESCRIPTION OF THE DRAWINGS

FIG. 1 shows a schematic diagram illustrating an embodiment of the system as described herein.

FIG. 2 is a perspective view of one embodiment of a device in accordance with the present disclosure.

FIG. 3 is a side view of one embodiment of a device in accordance with the present disclosure.

FIG. 4 is a front view of one embodiment of a device in accordance with the present disclosure.

FIG. 5 is a perspective view of one embodiment of an interactive care and personal engagement facilitation device in accordance with the present disclosure.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

In various embodiments, the system and each communications or computing device has a processor and an associated memory storing instructions that, when executed by the processor, cause the processor to perform operations as described herein. It will be appreciated that reference to “a”, “an” or other indefinite article in the present disclosure encompasses one or more than one of the described element. Thus, for example, reference to a processor encompasses one or more processors, reference to a memory encompasses one or more memories, reference to a monitor encompasses one or more monitors, reference to a clamp encompasses one or more clamps and so forth.

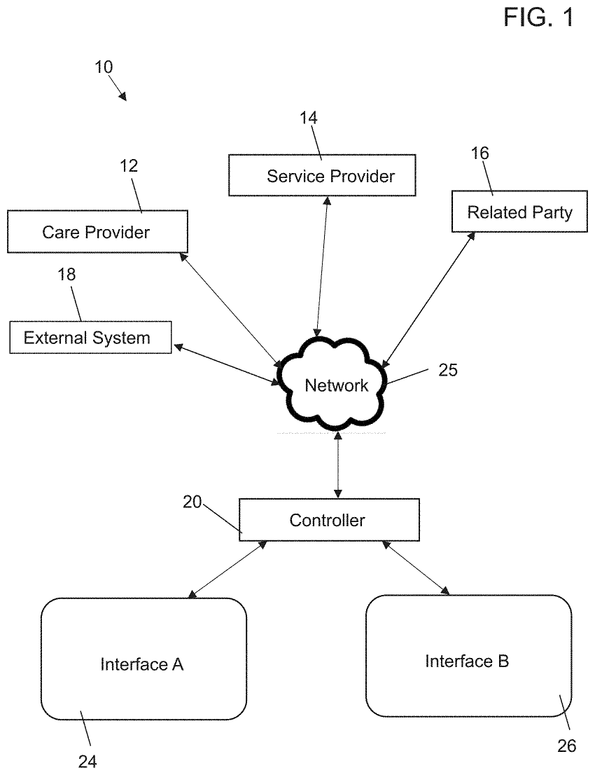

As shown in FIG. 1, embodiments of the system 10 disclosed herein can include a central controller 20 in communication with various users and/or devices over a network 25. The users may include a care provider using a care provider device 12, a service provider using a service provider device 14, a related party such as a family member using a related party device 16 and an external system device 18. The central controller 20 is further operable to communicate with one or more interfaces 24, 26 that may permit communication and interaction with an end user such as a resident at a senior living or other caregiving facility. In various embodiments, the interfaces 24, 26 are mounted to a portable device 30 to support various uses such as disclosed herein. In alternative embodiments, the central controller 20 is provided as multiple controllers, with each controller in communication with a specific interface such as interfaces 24, 26. The central controller 20 permits operation of the device remotely, such as by a care provider accessing the care provider device 12 and issuing commands to the central controller, for example. The interfaces 24, 26, with or without one or more controllers 20, can be considered as interactive care and personal engagement facilitation devices.

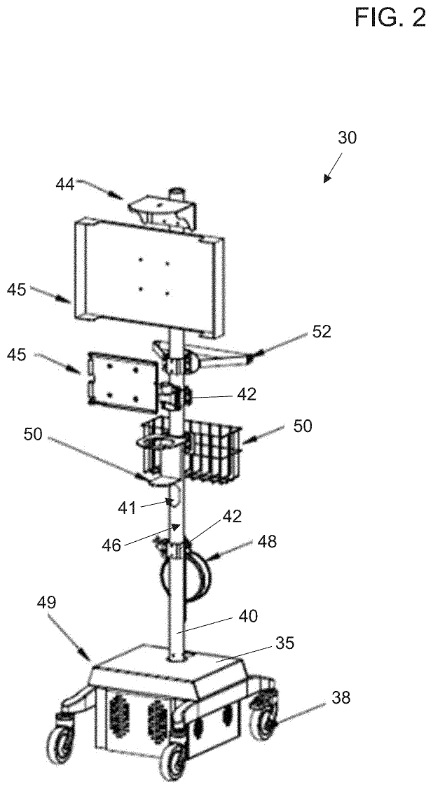

As shown in FIGS. 2 through 5, the portable device 30 can include a stand 35 mounted on lockable casters 38 or other rolling devices to permit the stand 35 to be moved along the ground or floor surface and locked in place upon arrival at a desired destination. A support pole 40 can be mounted to the stand 35 and provided with mounting hardware to support desired equipment at multiple heights along the pole. For instance, the mounting hardware can include one or more clamps 42 securable to the pole, wherein various types of equipment can be secured to the clamps. Such equipment can include, for example, a camera mount 44 for receiving a camera 55 to capture images of a desired person or subject, one or more monitor shrouds or brackets 45 to support a video monitor, tablet or similar devices, a holder for a power cord 48, one or more baskets or other personal item retainers 50, a handle 52 for moving the stand and other devices. It will be appreciated that the camera 55 may also be mounted to one of the interfaces 24, 26 and not directly to the pole 40. Personal items may include books, antiseptic wipes, tissues, headphones 51, writing utensils such as stylus devices for interface 26 and other such items. It will be appreciated that multiple headphone pairs can be employed and connected to the system disclosed herein so as to reduce noise and potential privacy concerns in environments where other parties may be within hearing range of the user and/or other participants or providers in communication with the user. It will further be appreciated that Bluetooth and/or other wireless technologies can be used with the headphones and other articles as necessary to avoid wired connections, which may sometimes interfere with other activities. In various embodiments wherein a computing device such as a laptop or tablet computer is secured to a bracket 45, such computing device acts as the central controller 20. The handle 52 can be movably secured around the pole 40 to facilitate ease of operation by different users. For example, the handle 52 may be positioned behind interface 26 for some users but may be moved higher or lower depending upon the user's height and/or other variables. In certain embodiments, one or more ports such as USB ports can be provided in communication with the controller, permitting certain external devices such as suitably equipped stethoscopes or other medical devices to be connected.

The camera 55 can be removable and can be an adjustable web camera to facilitate up-close health care professional capabilities, according to various embodiments. In various embodiments, any issues detected via the camera 55 can be addressed by the remotely connected individual. For example, a nurse practitioner who senses that an emergency or non-emergency is occurring based upon viewing the end user through the camera can notify appropriate third parties to address the event.

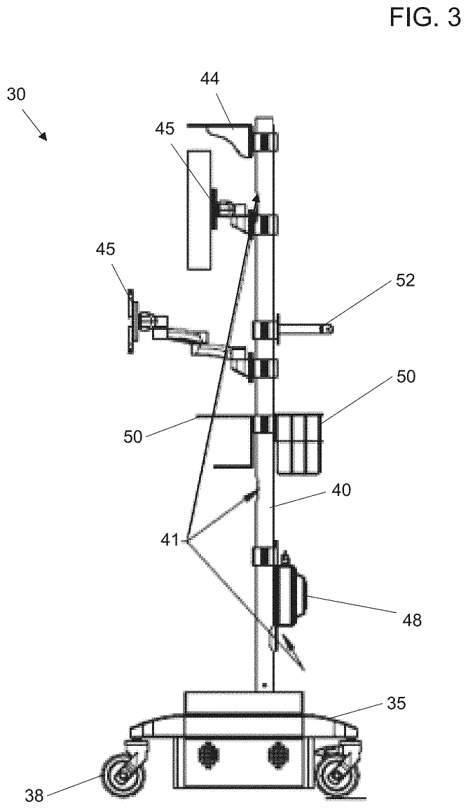

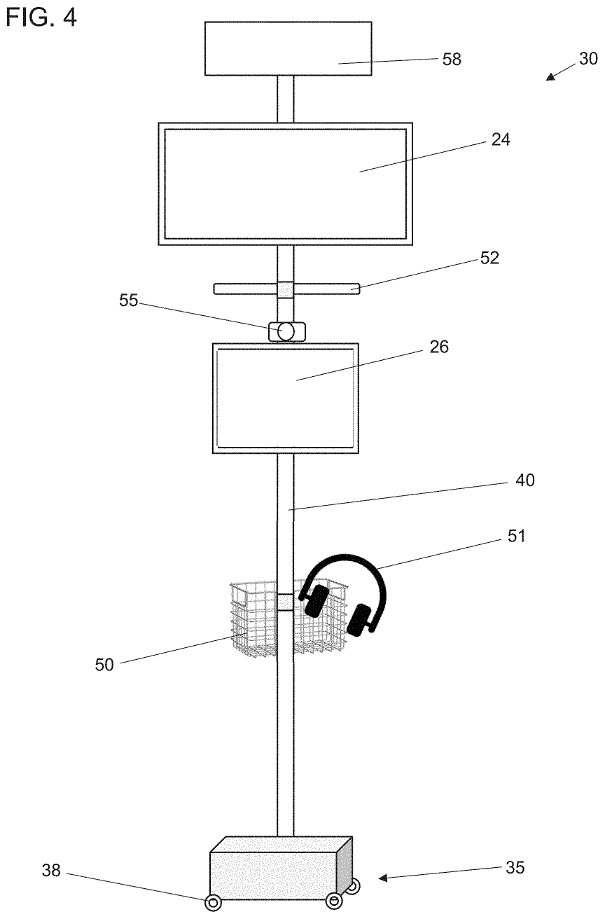

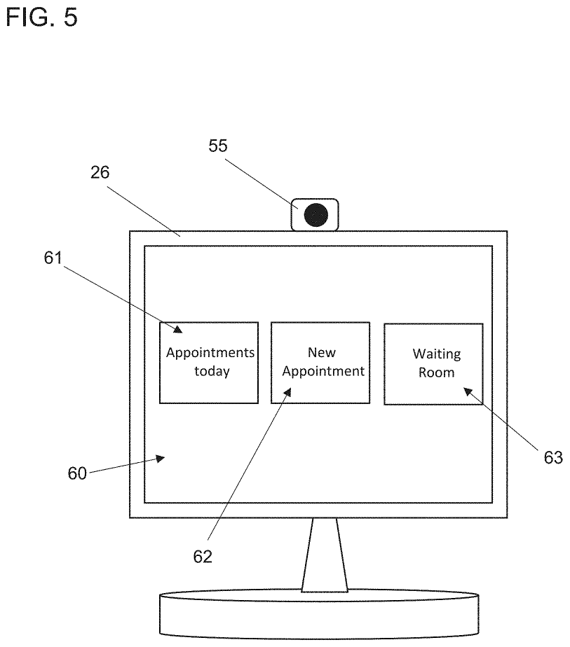

In various embodiments, the pole is 40 provided with one or more openings 41 in its outer wall 46 for managing electrical and communications wiring, for example. A power supply 49 such as a battery may be provided and maintained on or under the stand 35 or on the pole 40. Alternatively, wiring can be secured to the device 30 and connected to a suitable external power source. FIGS. 4 and 5 show the interfaces 24, 26 secured to the monitor brackets 45, which are shown in FIGS. 2 and 3. It will be appreciated that the interfaces 24, 26 can be arranged in side-by-side (FIG. 5) or stacked (FIG. 4) configuration, among other configurations. In various embodiments, one interface can be secured to the approximate center of the pole and can be moved left, right, up and down, whereas the second interface is secured to an articulating arm secured to the pole. The articulating arm can be moved backwards, forwards, up, down, left, right and generally in any direction. The flexible positioning of the interfaces permits the device to be adapted to a variety of environments and available space. In some embodiments, additional surface real estate is provided in the form of a sign or support bracket for a sign such as at 58 in FIG. 4 to permit advertising or other messaging on the device 30. For example, a health plan provider or health care service may wish to place their name, trademark or other information on the sign 58 to gain exposure to existing or potential new customers in a permanent fashion, as opposed to displaying pertinent material on monitors which may be readily turned on or off.

In various embodiments, the interfaces 24, 26 are touchscreen interfaces, wherein the first interface 24 is a slave monitor and the second interface 26 is a tablet computing device. Each interface 24 and 26 provides a visual display. In various embodiments, the interfaces 24, 26 include one or more speakers such that the user can hear audible sounds (e.g., voices) from the system and one or more microphones so that the user's voice and any sounds from around the device 30 can be heard, recorded and/or transmitted. The central controller 20, which may be embodied as a tablet computing device with touch screen interface 26, can have any number of computer applications running on it. For instance, one or more scheduling applications and video conferencing applications may be run via the central controller 20. Such applications may be stored in a memory of the central controller or stored in the “cloud” and operable by the processor of the central controller 20. In various embodiments, an application is provided and operable by the central controller to allow meeting scheduling where meetings are created, with invitations sent to the participants (which may be family, healthcare professionals, etc., as described herein). When it is time for the meeting, the application opens up a secure point-to-point video communication with each invited party. It will be appreciated that each video call can be recorded and stored securely. Further, electronic documentation can be shared using a screen share with all video conference participants, and all documents electronically signed can be stored securely in a document management program or system operable by the central controller 20 or as an external system accessible via the central controller 20. In various embodiments, interface 24 is a large screen monitor for easy viewing by a user and/or patient during appointments.

The interface 26 can be employed as part of device 30 or independently as a standalone device. For example, interface 26 may be employed as a portable central healthcare access portal in a user's home or other location. As shown in FIG. 5, interface 26 includes a display 60 which can display a variety of viewable output such as healthcare data, live video and input mechanisms. For example, input box 61 can be a touchscreen, mouse-over or clickable icon that, upon being selected by a user, retrieves stored information regarding the user's appointments for a given time frame, such as daily, weekly or other time frame. Input box 62 can be a touchscreen, mouse-over or clickable icon that, upon being selected by a user, permits the user to set up a new appointment. Input box 63 can be a touchscreen, mouse-over or clickable icon that, upon being selected by a user, places the user in a virtual waiting room for an appointment and further informs the service provider with whom the appointment exists that the user is waiting for the appointment to begin. The healthcare data to be displayed can include electronic documentation such as a health plan application, an existing health plan, a pre-authorization form, a health risk assessment, a wellness visit questionnaire, user-related diagnostic information such as blood pressure, blood test results, heart rate, glucose levels and other user-specific healthcare information.

It will be appreciated that the present system 20 and device 30 facilitate remote access, control and interaction with users such as individuals who may otherwise be isolated with limited personal engagement, and can be particularly helpful when there is limited access to facilities which may house such individuals. For example, senior living facilities may need to limit access by healthcare professionals, family members or other responsible parties, health plan staff and other individuals and service providers. If the location where the user resides has limited access due to remote location, contagious disease or other reason, the presently disclosed system and device permits such users to access necessary or desired individuals when and where needed. As the device is portable, the user may access others remotely in their own personal room, in a common area, outdoors or generally any location where the user and the device may co-reside. In various embodiments, users can employ one or more of the touchscreen interfaces to see or electronically sign documents. An outside party in remote communication with the user can also notarize the user's signature as necessary. Regardless of activity, embodiments of the present disclosure enable relatives or other responsible parties to see the user and participate in planned visits with health practitioners and other outside parties. Among other uses, the system can be employed to set meetings and calendar appointments for the end user.

In embodiments, the present system and device can be employed as an interactive sales kiosk for presenting plan and sales information, or as an interface for service providers to complete pre-authorizations, health risk assessments, annual wellness visit questionnaires or to share electronic medical records (EMRs) with other clinicians who may be joining by video conference, for example. The system and device can also be used in assisted living or independent living homes as a device for enabling a member to connect to a healthcare professional or to monitor vital signs such as heart rate, glucose levels, blood pressure and other signs. It will be appreciated that health plan employees or clinicians can control which software applications can be run on the tablet computing device. In various embodiments, the device can be operated remotely by a remote party or by artificial intelligence for movement and interactive functionality.

Example Use Case 1:

In one example, a healthcare professional agrees on a date and time to conduct a plan visit with a facility resident/member and the resident's responsible party (e.g., family member) using the system and device as described herein. In various embodiments, the controller 20 includes a scheduler for the date and time to be entered and reminders can be sent to the parties, as appropriate. At or before the appointed date and time, the controller 20 can send a communication (e.g., email or text message) with the appointment information and a link to be used for the video communication. The professional may use device 12 to access the controller 20 or may access the controller 20 directly using a controller interface. The responsible party may receive the communication(s) and access the meeting via responsible party device 16. As needed, the professional may clean the device prior to bringing it to the resident's location for the meeting. Once the device is in the desired location, the professional or the user can turn the device on and launch the required videoconferencing software. In various embodiments, the resident user can be provided with a headset to facilitate hearing and speaking during the meeting. Once the responsible party joins the video meeting (such as by clicking on the previously communicated link), the meeting between the resident, in-person healthcare professional and remote responsible party can occur.

Example Use Case 2:

In another example, a healthcare professional agrees on a date and time to conduct a plan visit with a facility resident/member and another healthcare professional (e.g., primary physician) using the system and device as described herein. In various embodiments, the controller 20 includes a scheduler for the date and time to be entered and reminders can be sent to the parties, as appropriate. At or before the appointed date and time, the controller 20 can send a communication (e.g., email or text message) with the appointment information and a link to be used for the video communication. The professional may use device 12 to access the controller 20 or may access the controller 20 directly using a controller interface. The remote service provider may receive the communication(s) and access the meeting via service provider device 14. As needed, the professional may clean the device prior to bringing it to the resident's location for the meeting. Once the device is in the desired location, the professional or the user can turn the device on and launch the required videoconferencing software. In various embodiments, the resident user can be provided with a headset to facilitate hearing and speaking during the meeting. Once the responsible party joins the video meeting (such as by clicking on the previously communicated link), the meeting between the resident, in-person healthcare professional and remote healthcare professional can occur. In such a situation, it will be appreciated that the in-person healthcare professional can share EMR screens, close-up videos or images of physical conditions of the resident and other needed information for the remote healthcare professional. Any necessary healthcare visit documentation can be recorded and stored via the controller, in various embodiments.

Example Use Case 3:

In another example, an external system or party 18 is in communication with the controller 20 and can provide a demonstration to a user accessing the device interface (24 or 26). The external system 18 can provide pre-established information, such as text, images and/or video suited to the requests of the user, and/or may provide live and/or interactive information in real-time based on the user's queries. As an example, a user can use the touchscreens to peruse health plan information and should the user so desire, the user may select a “contact me” button or icon to connect the user with a live agent video conference. Alternatively, such action may send an email to the sales organization for follow-up.

Example Use Case 4:

In another example, an external system or party 18 is in communication with the controller 20 and can provide interactive forms for a user to self-sign by accessing the device interface 26. An outside party such as a family member accessing the controller 20 via device 16 can assist the user in reviewing and approving the required forms. A service provider can simultaneously access the controller 20 via device 14 to interact with the user and/or the family member. In various embodiments, the user can see the necessary document to be signed on the interface 24 while seeing the service provider, family member or both on the monitor 26. In various embodiments, the user may engage in a self-enroll process provided as part of the programming available through the central controller 20. If the user reviews the health plan details for a given plan and wishes to enroll, the system can guide the user through the full enrollment experience without the need for a remote or in-person agent at all.

The present system, device and method can thus receive one or more requests from a user, where the request(s) can be for live video communication with a remote party and/or can be for a display of healthcare data, for example. In response to the different requests, the system can present a live video display of the remote party on the first interactive care and personal engagement facilitation device (e.g., 24) and present a display of the healthcare data on the second interactive care and personal engagement facilitation device (e.g., 26). The remote party can be a healthcare professional, a party responsible for care and/or payment related to the user, a service provider and a document system. In various embodiments as described elsewhere herein, the system can share the healthcare data with the remote party accessing the system. For example, if a user is completing a wellness visit questionnaire and the user's family member is remote from the user but in communication with the user via interface 24, the user's family member can view the questionnaire on the family member's own computing device and can assist the user in completing the questionnaire, where the user may complete the questionnaire via interface 26. The user can also sign (e.g., electronically) forms and other documentation via interface 26.

The above-described embodiments of the present disclosure may be implemented in accordance with or in conjunction with one or more of a variety of different types of systems, such as, but not limited to, those described below. It will be appreciated that the present system, device and method is not to be considered as limited to a healthcare environment, as the elements and programming described herein can be employed in a wide variety of environments.

The present disclosure contemplates a variety of different systems each having one or more of a plurality of different features, attributes, or characteristics. A “system” as used herein can refer to various configurations of: (a) one or more remote computing devices; (b) one or more personal computing devices, such as desktop computers, laptop computers, tablet computers, personal digital assistants, mobile phones, and other mobile computing devices; and (c) one or more of (a) and (b).

In certain embodiments in which the system includes a personal computing device in combination with a remote computing device, the personal computing device includes at least one processor configured to transmit and receive data or signals representing events, messages, commands, or any other suitable information between the personal computing device and the remote computing device. The personal computing device can further include input/output mechanisms such as a keyboard, mouse and display or monitor. The processor of the personal computing device is configured to execute the events, messages, or commands represented by such data or signals in conjunction with the operation of the personal computing device. Moreover, the processor of the remote computing device is configured to transmit and receive data or signals representing events, messages, commands, or any other suitable information between the remote computing device and the personal computing device. The processor of the remote computing device is further configured to execute the events, messages, or commands represented by such data or signals in conjunction with the operation of the remote computing device.

In embodiments in which the system includes a personal computing device configured to communicate with a remote computing device through a data network, the data network is a local area network (LAN), a wide area network (WAN), a public network such as the Internet, or a private network. The remote computing device and the personal computing device are configured to connect to the data network or remote communications link in any suitable manner. In various embodiments, such a connection is accomplished via: a conventional phone line or other data transmission line, a digital subscriber line (DSL), a T-1 line, a coaxial cable, a fiber optic cable, a wireless or wired routing device (e.g., WiFi), a mobile communications network connection (such as a cellular network or mobile Internet network), or any other suitable medium.

It will be appreciated that any combination of one or more computer readable media may be utilized. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing, including a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

As will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon.

It will be appreciated that all of the disclosed methods and procedures herein can be implemented using one or more computer programs or components. These components may be provided as a series of computer instructions on any conventional computer-readable medium, including RAM, SATA DOM, or other storage media. The instructions may be configured to be executed by one or more processors which, when executing the series of computer instructions, performs or facilitates the performance of all or part of the disclosed methods and procedures.

Unless otherwise stated, devices or components of the present disclosure that are in communication with each other do not need to be in continuous communication with each other. Further, devices or components in communication with other devices or components can communicate directly or indirectly through one or more intermediate devices, components or other intermediaries. Further, descriptions of embodiments of the present disclosure herein wherein several devices and/or components are described as being in communication with one another does not imply that all such components are required, or that each of the disclosed components must communicate with every other component. In addition, while algorithms, process steps and/or method steps may be described in a sequential order, such approaches can be configured to work in different orders. In other words, any ordering of steps described herein does not, standing alone, dictate that the steps be performed in that order. The steps associated with methods and/or processes as described herein can be performed in any order practical. Additionally, some steps can be performed simultaneously or substantially simultaneously despite being described or implied as occurring non-simultaneously.

It will be appreciated that algorithms, method steps and process steps described herein can be implemented by appropriately programmed computers and computing devices, for example. In this regard, a processor (e.g., a microprocessor or controller device) receives instructions from a memory or like storage device that contains and/or stores the instructions, and the processor executes those instructions, thereby performing a process defined by those instructions. Furthermore, aspects of the present disclosure may take the form of a computer program product embodied in one or more computer readable media having computer readable program code embodied thereon.

Computer program code for carrying out operations for aspects of the present disclosure may be written in any combination of one or more programming languages, including an object oriented programming language such as Java, JavaScrypt, Scala, Smalltalk, Eiffel, JADE, Emerald, C++, C #, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on a user's computer, partly on a user's computer, as a stand-alone software package, partly on a user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

1. A system, comprising:
a first interactive care and personal engagement facilitation device comprising a first visual display and a speaker;
a second interactive care and personal engagement facilitation device comprising a second visual display comprising a touch screen interface; and
a controller comprising a processor and a memory storing instructions that, when executed by the processor, cause the processor to:
receive a first request from a user for live video communication with a remote party;
receive a second request from the user for a display of data; and
in response to the first and second requests, present a live video display of the remote party on the first interactive care and personal engagement facilitation device and present a display of the data on the second interactive care and personal engagement facilitation device.

2. The system of claim 1, wherein the remote party is one of: a healthcare professional, a health plan enrollment professional, a health plan coordinator and a family member.

3. The system of claim 1, wherein the display of data comprises a health plan application or a health plan.

4. The system of claim 1, wherein the instructions, when executed by the processor, further cause the processor to enable control of the first interactive care and personal engagement facilitation device or the second first interactive care and personal engagement facilitation device by the remote party.

5. The system of claim 1, wherein the live video communication comprises a secure point-to-point video communication.

6. The system of claim 1, wherein the data comprises electronic documentation, and wherein the instructions, when executed by the processor, cause the processor to share the electronic documentation with the remote party.

7. The system of claim 1, wherein the instructions, when executed by the processor, cause the processor to receive a signature from the user via the touchscreen interface.

8. The system of claim 1, wherein the data comprises one of: a pre-authorization form, a health risk assessment, a wellness visit questionnaire and an electronic medical record.

9. The system of claim 1, wherein the data comprises vital signs monitoring data comprising a heart rate, a glucose level and a blood pressure.

10. A mobile interactive care and personal engagement device, comprising:
a movable stand;
a support pole secured to the stand; and
at least two interactive care and personal engagement facilitation devices secured to the support pole, from the group consisting of: a video camera, a video display and an interactive touch screen.

11. The device of claim 10, wherein the at least two interactive care and personal engagement facilitation devices further comprise a controller.

12. The device of claim 10, wherein the controller comprises a processor and a memory storing instructions that, when executed by the processor, cause the processor to:
receive a first request from a user for live video communication with a remote party;
receive a second request from the user for a display of data;
in response to the first and second requests, present a live video display of the remote party on the first interactive care and personal engagement facilitation device and present a display of the data on the second interactive care and personal engagement facilitation device.

13. The device of claim 12, wherein the remote party is one of: a healthcare professional, a health plan enrollment professional, a health plan coordinator and a family member.

14. The device of claim 12, wherein the display of data comprises a health plan application or a health plan.

15. The device of claim 12, wherein the instructions, when executed by the processor, further cause the processor to enable control of one of the at least two interactive care and personal engagement facilitation devices by the remote party.

16. The device of claim 12, wherein the live video communication comprises a secure point-to-point video communication.

17. The device of claim 12, wherein the data comprises electronic documentation, and wherein the instructions, when executed by the processor, cause the processor to share the electronic documentation with the remote party.

18. The device of claim 12, wherein the instructions, when executed by the processor, cause the processor to receive a signature from the user via the touchscreen interface.

19. The device of claim 12, wherein the data comprises one of: a pre-authorization form, a health risk assessment, a wellness visit questionnaire and an electronic medical record.

20. The device of claim 12, wherein the data comprises vital signs monitoring data comprising a heart rate, a glucose level and a blood pressure.
